(19) United States
(12) Patent Application Publication
(10) Pub. No.: US 2023/0277086 A1
(43) Pub. Date: Sep. 7, 2023

(54) SYSTEM AND METHOD FOR THE ASSESSMENT OF THE BIOMECHANICAL RISK FROM MANUAL HANDLING OF LOADS

(71) Applicant: ALMA MATER STUDIORUM - UNIVERSITÀ DI BOLOGNA, Bologna (IT)

(72) Inventors: Roberta BONFIGLIOLI, Bologna (IT); Elena BORELLI, Bologna (IT); Beatrice FRABONI, Bologna (IT); Marta TESSAROLO, Bologna (IT); Lorenzo CHIARI, Bologna (IT); Francesco VIOLANTE, Bologna (IT)

(73) Assignee: ALMA MATER STUDIORUM - UNIVERSITÀ DI BOLOGNA, Bologna (IT)

(21) Appl. No.: 18/040,658

(22) PCT Filed: Aug. 4, 2021
(86) PCT No.: PCT/IB2021/057163

§ 371 Date: Feb. 6, 2023

(30) Foreign Application Priority Data
Aug. 5, 2020 (IT) 102020000019315

(51) Int. Cl. A61B 5/103; A61B 5/00

(52) U.S. Cl. CPC A61B 5/1036; A61B 5/6807; A61B 5/7275; A61B 2503/20

(57) ABSTRACT

A system and method for assessing biomechanical risk in the context of handling heavy loads in workplaces. The system and method are applicable during a normal working activity for acquisitions lasting a work shift. The acquired data are then analysed in relation to regulatory references and international standards for the assessment of the biomechanical risk from manual handling of loads.

### CROSS-REFERENCES TO THE RELATED APPLICATIONS

The application is a national stage entry of PCT/IB2021/057163 filed on Aug. 4, 2021, which claims priority to patent applications No. IT 102020000019315 filed on Aug. 6, 2020, the entire contents of which are incorporated herein by reference.

### TECHNICAL FIELD

The present invention relates to the field of biomechanical risk assessment from manual handling of loads.

### BACKGROUND

Biomechanics is the application of the principles of mechanics to the human body. In particular, biomechanics analyses the behaviour of the organism's structures when subjected to static or dynamic stresses (Chaffin D B, Gunnar Andersson G B J, Martin B J. Occupational Biomechanics. John Wiley & Sons, 4th edition, 2006).

Disorders and diseases affecting the musculoskeletal structures due to biomechanical overload can be caused by work activities characterized by a constant functional commitment of the body district concerned. The development of these conditions is mainly linked to the manual handling of loads, to manual work that requires strength, speed and continuity of movements, to the assumption of incongruous postures and exposure to vibrations. Epidemiological evidence suggests that manual handling of too heavy or improperly lifted loads can lead to the development of back pain or, in some cases, to injury to the vertebral structures.

The risk assessment of numerous activities that involve the manual handling of loads is fundamental in the field of safety in the workplace, an area in which it is essential to accurately evaluate the number and weight of each load moved by a worker during his activity, as well as the duration and frequency of manual load handling activities.

Nowadays, risk assessment is performed through empirical observation by an assessor, subjective assessment by workers, employee reporting or video analysis, or a set of such non-standardized assessment techniques. This involves additional tasks for the worker in order to monitor his activity, which can alter the same work procedures that are the subject of the evaluation itself. Furthermore, the techniques mentioned above are often not particularly precise and not reliable, not offering a complete and objective description of the biomechanical load to which the worker is subjected.

When the duration of the handling tasks covers a large portion of the work shift and when the frequency of handling is not regular, an observational evaluation requires an extremely large sampling time by the evaluator: this both in case of direct observation and in case of deferred observation by video recordings. Furthermore, the operational variability present between different subjects makes it essential to observe more than one worker with a considerable waste of resources; the same is experienced with regard to the variability of the observer. Finally, it should be added that workplaces are not always easily accessible for an external evaluator, such as for example a surgery room.

Hence, the need for devices capable of recording the manual handling of loads for the duration of an entire work shift, under different conditions of activity, for more than one worker. In this way the biomechanical risk assessment will be able to reach a higher degree of accuracy with respect to the real handling conditions and their variation.

The possibility that the aforementioned devices for recording handling activities are wearable also allows their use in different working conditions.

Several patent documents report wearable pressure sensors that can be incorporated into shoes, socks or insoles.
WO2017120063 Footwear with pressure sensor and collection system
WO2016367191 Sensor systems applicable to footwear or clothing, for monitoring contact, force, pressure and/or cut at or near body surfaces.
CN106307775 Foot pressure measurement system, using smart sneakers.
CN106447568 Method for detecting position, gait and relating gait correction. Intelligent Foot Biological Health Information Management System.
WO2018MX00056 System for the early diagnosis of diabetic foot syndrome.

These documents describe pressure sensor systems capable of assessing the posture of the foot and providing feedback to the user or to an external person/doctor. The main objective of the described devices is to correct posture, walking or running, or they have been developed for medical applications such as the detection of diabetic foot syndrome.

Also in the clinical setting, applications are described that use sensorized insoles and also inertial sensors, for the evaluation and recognition of postural attitudes and activities performed (for example walking, running, stairs) (Ngueleu A M, Blanchette A K, Maltais D, Moffet H , McFadyen B J, Bouyer L, Batcho C S. Validity of Instrumented Insoles for Step Counting, Posture and Activity Recognition: A Systematic Review. Sensors (Basel). 2019 May 28; 19 (11)).

Systems equipped with load cells applied to the sole of shoes and associated with inertial sensors are also known. They were used to estimate the load on the L5 S1 segment of the backbone. However, these are devices applicable only in a laboratory context (Faber G S et al. Estimating 3D L5/S1 moments and ground reaction forces. J Biomech. 2018 March 21; 70: 235-241)

Again, in the laboratory context, the use of insoles for the estimation of the lifted load was described, in static and dynamic conditions, but only for short periods and without the possibility of integrating the data with a measurement of the distance traveled (Ellegast R, Kupfer J, Reinert D. Load weight determination during dynamic working procedures using the pedar foot pressure distribution measuring system. Clin Biomech (Bristol, Avon). 1997 April; 12 (3): S10-S11).

All existing systems are therefore focused on analysing the distribution of contact pressures exerted by the body on the sole of the foot.

If not specifically excluded in the detailed description that follows, what is described in this chapter is to be considered as an integral part of the detailed description.

### SUMMARY

The purpose of the present invention is to provide a system capable of assessing whether the activities that are performed by an operator, for example the handling of heavy loads, can configure a risk of biomechanical overload for the operator's body.

The basic idea of the present invention is to detect variations in pressure on the sole of the foot, monitored during the performance of an activity, to objectively assess the biomechanical risk associated with this manual handling of loads.

More specifically, through the use of this system, the presence of an additional load with respect to the weight of the operator subject to measurement is identified and the movement of the load is monitored, including the duration and frequency of lifting, lowering and transport activities, associated with the measurement of the distances travelled, in order to objectively evaluate the biomechanical risk associated with lifting heavy objects.

Advantageously, thanks to the present invention it is possible to identify the average weight of the loads of significant mass lifted by an operator, the number of times the loads are lifted and the distances travelled while the loads are supported.

It is also possible to perform an assessment on the working environment by repeating the aforementioned monitoring on several operators, thus improving the accuracy of the assessment of the risk of diseases affecting the musculoskeletal system in a predetermined working environment.

The proposed system includes an insole equipped with a predetermined number of pressure sensors, preferably eight, arranged at the forefoot and heel. The device provides for information on the number of objects of significant weight, typically greater than 3 kg, lifted by the monitored operator, their mass, frequency and duration of their handling.

Advantageously, the insoles are adaptable to the different dimensions of the feet.

The sensors are not directly in contact with the skin and sweat and can be worn multiple times and by different users.

Another purpose of the invention is a method for monitoring the weight variation of a moving operator, due to the lifting of a load. According to the present invention, in addition to the weight variation due to the lifting of a heavy load, the number of times in which said weight variation occurs, in a defined period of time, is also monitored, giving as output a weight measurement, with an error of less than 10%, of the number of loads handled in the defined time frame and the duration of the handling activities.

The system includes processing means configured to acquire the aforementioned data, organize them and determine the type of weights handled, the frequency and duration of the handling and calculate a biomechanical risk expressed through a numerical index correlated to the risk of developing back pain, taking into account the anthropometric characteristics of the monitored operator.

Further purposes will become clear from the description of the invention that follows and from the dependent claims, which form an integral part of this description.

### BRIEF DESCRIPTION OF THE DRAWINGS

The features and advantages of the invention will appear evident from the detailed description that follows, made with reference to the attached drawings, provided purely by way of non-limiting example, in which:

FIG. 1 shows a conceptual scheme of components defining the system object of the present invention;

FIGS. 2 and 3 show responses processed by the system in the static and dynamic phase, respectively, relative to a plurality of sensors associated with an insole shown in FIG. 1;

FIG. 4 shows signals representative of load measurements carried out by means of the system of the preceding figures;

FIG. 5 shows an example of processing, measurements by means of a block diagram, of signals relating to the aforementioned plurality of sensors;

FIG. 6 is a block diagram showing a schematic configuration of a biomechanical risk assessment mode according to an embodiment of the present invention.

In the context of this description, the term “second” component does not imply the presence of a “first” component. These terms are in fact used as labels to improve clarity and should not be understood in a limiting way.

The elements and features illustrated in the various preferred embodiments, including the drawings, can be combined with each other without however departing from the scope of this application as described below.

### DETAILED DESCRIPTION OF THE EMBODIMENTS

In the context of the present invention, the terminology “biomechanical risk” is intended to indicate those conditions in which, during the performance of a work activity, elements of the task called “biomechanical factors” are present, i.e. forces applied to parts of the body, or developed by the body, in order to perform a job. Among the activities that most frequently may require the evaluation of these factors is the manual handling of loads.

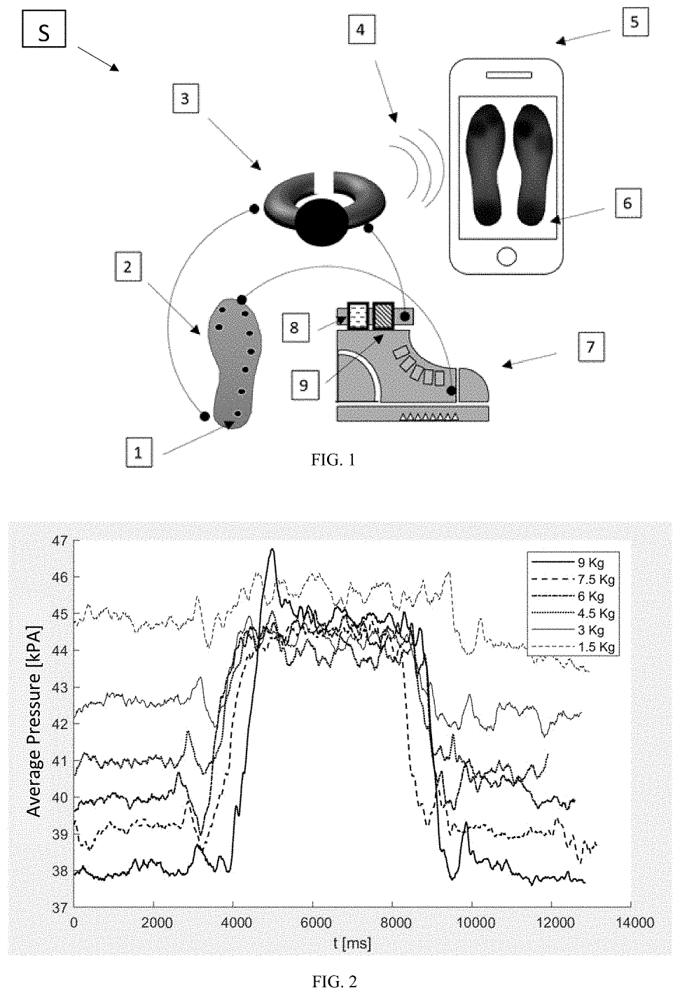

With particular reference to FIG. 1, an example of system S according to the invention comprises the elements identified in the following legend:

Insole 1

Pressure sensors 2

Acquisition electronics 3

Data interface means 4

Analysis and processing device 5

User interface 6

Shoe 7

Accelerometer 8

Gyroscope 9

A plurality of pressure sensors 2 is associated with an insole that can be extracted from a shoe 7.

FIG. 1 shows a work shoe, but it must be taken into account that the insole can be installed in any shoe.

It can also be envisaged that the insole is stably integrated into a shoe, making the same shoe separable from the acquisition electronics 3.

In the case of an insole that can be separated from the shoe, it is evident that this is indirectly wearable, in the sense that it remains in contact with the human body because it is bound by the shoe in a sandwich configuration.

One or more pressure sensors are preferably associated with the insole, for example but not limited to eight sensors 2, which detect the pressure to which the insole is subjected when worn, so as to be able to measure the weight and its distribution.

The sensors associated with the insole are operatively connected to the electronics 3 for acquiring the signals generated by the sensors, for example by means of a flat cable.

The acquisition electronics 3 comprises connection means for fixing the acquisition electronics 3 to the ankle of an operator or to the neck of the shoe.

The acquisition electronics comprises a wireless interface 8, for example of the Bluetooth type or similar, through which the acquisition electronics 3 transmits the signals acquired by the plurality of sensors 2 to a data analysis and processing device 5, which can be a smartphone, a tablet or a suitably configured computer, for example by means of an application.

The recipient of the measurements can be the same operator, or the measurements can be forwarded to a remote computer.

Preferably, both shoes are equipped with a sensorized and interfaced insole 2, by means of the relative acquisition electronics 3 to the analysis and processing device 5.

At the beginning of a recording session, each insole is synchronized with a unique ID with the analysis and processing device 5 through the relative electronics 3 and integrated wireless interface. The technology that allows data transmission is Bluetooth with an optimized scanning speed to extend the battery life for a period of time at least equal to a daily work session.

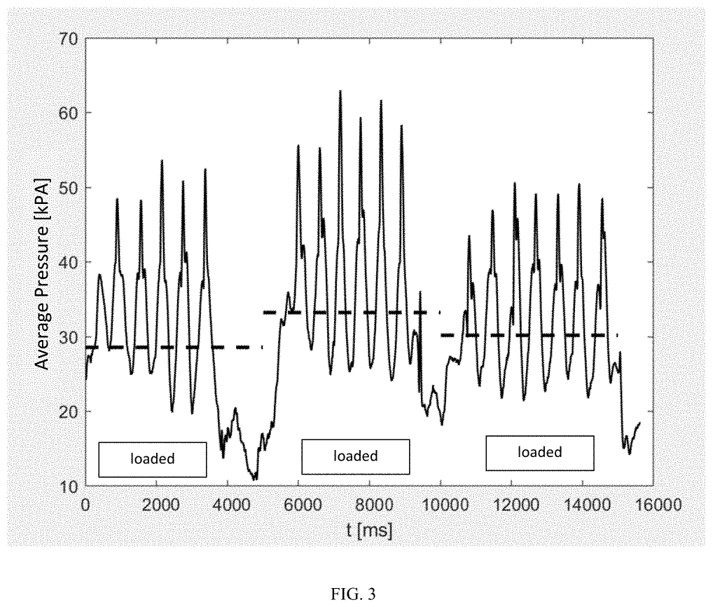

The biomechanical risk assessment system of the invention, for example shown in FIG. 1, is configured to record a load value lifted by the operator, when this value exceeds a predetermined threshold considered irrelevant for the purposes of assessing the biomechanical risk: this threshold can be for example 3 kg. FIG. 2 shows the signal obtained while carrying out a static test with different weights. The load is lifted after four seconds from the start of the test, is held in the hand for 5 seconds and is then stored away. The three phases are clearly distinguished for weights over 3 kg: in other words, the system is able to appreciate weight variations over 3 kg.

FIG. 3 shows the signal obtained in a dynamic test, that is, while the operator walks, in conditions “without additional load”, “with additional load” and still “without additional load”. The solid line indicates the raw data relating to the load per unit area of the insole in which the contribution of walking on the signal is evident. The straight lines, horizontal and shown in broken lines, indicate average values of the load per unit surface area of the insole, obtained by applying a high pass filter.

Preferably, the pressure sensors 2 are of the capacitive type, that is to say, each comprises two metal elements/electrodes separated by a dielectric material. Each pressure sensor is protected/encapsulated between two protective layers, preferably of polyethylene to protect it from moisture and sweat. Sensor 2 thus made is about 2 mm thick and defines an area of about 1 cm2.

Each sensor 2 generates an electrical signal representative of a pressure variation applied to the same sensor.

In the context of the invention, by pressure applied to the sensor, it is meant the force applied by the operator per unit of surface of the insole. From the variation of the capacity, it is possible to derive the applied pressure and then, by multiplying by the area of the insole, which in the example has an area of 198 cm2, to obtain the overall measured weight. Obviously, the area of the insole depends on the size of the foot of the monitored operator.

When the operator lifts a load, the weight of the load is defined by subtracting the weight measured when the operator is unloaded (tare), in order to identify by difference the weight of the object, or additional load, lifted. This obviously occurs when the measured load is greater than the aforementioned threshold, for example by 3 kg.

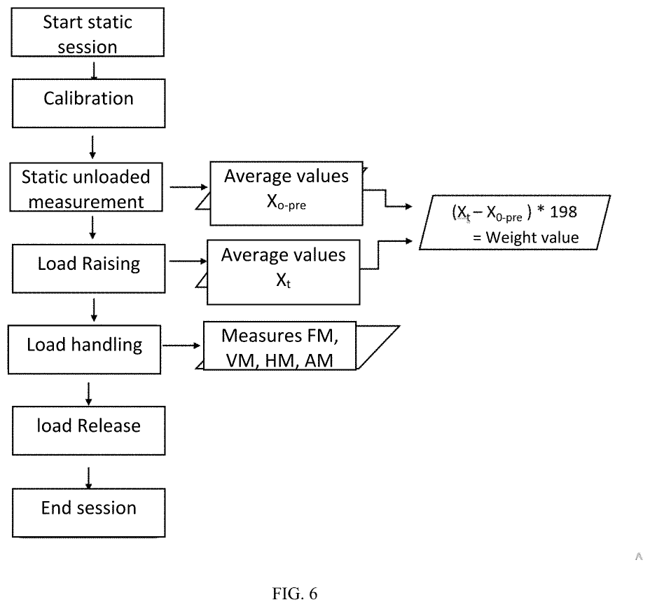

Following the block diagram shown in FIG. 6, the measurement session begins with a calibration that is carried out by means of a measurement at rest for 5 s without load, in order to identify an XO value calculated as the average of the signal of all the sensors of a single insole.

The weight (P) of a transported load is calculated as

P=(Xt−X0)/198 cm2

where Xt is the pressure measurement at time t and 198 cm2 the area of each insole. A weight P greater than 3 kg, i.e. 1.5 kg for each foot/insole is recognized as a load.

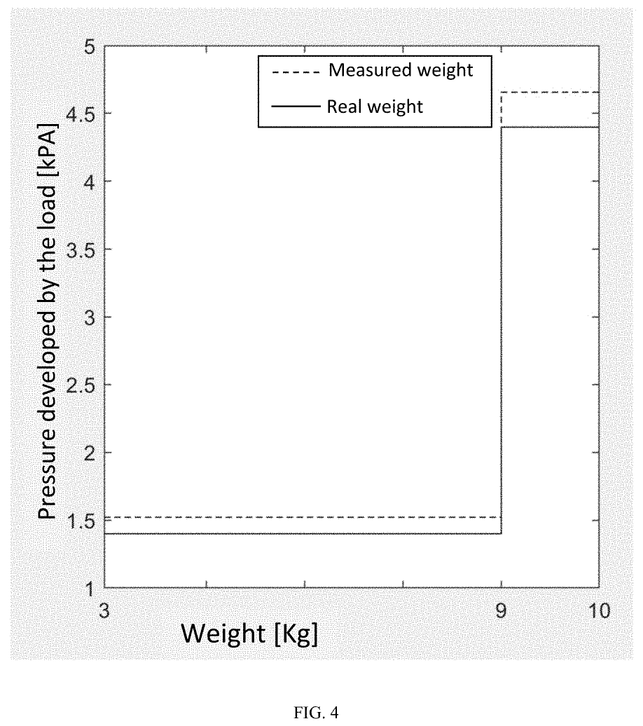

The tolerance in the weight measurement is about 10%, as can be appreciated from FIG. 4, which shows the comparison between the measurement of the load estimated through this system and the real weight of the load that weighs on both feet of the monitored operator.

This tolerance depends on the accuracy of the sensors and their arrangement within the insole.

Obviously, since the system S includes two insoles, right and left, the electronics 3 provides information on the weight of the load handled by the respective insole, on the duration of the movement and on any path travelled by the worker during handling.

Preferably, the electronics 3 is inserted in a container of limited dimensions 4×3×2 cm, and is waterproof as well as easily wearable. The impermeability allows easy sanitization of the electronics 3 in order to be used on multiple monitored operators.

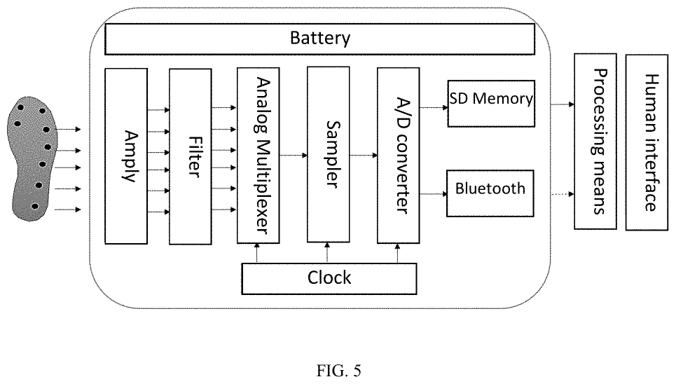

The electronics 3, as shown in detail in FIG. 5, includes components known per se, including elements for amplifying the electrical signals acquired by the sensors 2, low-pass and high-pass filters, a multiplexer and an analog/digital converter. Furthermore, the acquisition unit comprises a battery, preferably lithium, a housing for an SD memory and a sufficiently large SD memory, for example 2 GB and the aforementioned wireless interface means (Bluetooth) towards the analysis and processing device 5. The system can, indeed, be configured to communicate with one or more analysis and processing devices 5.

The processing analysis device 5 is configured to perform an adequate analysis of the acquired signals, by executing specific algorithms for the purposes of risk assessment, as well as to monitor the correct functioning of the system S itself.

With reference to FIG. 5, the electronics 3 includes
Differential amplifier to measure the capacitance of each pressure sensor 2
SD memory for recording data for long sessions, for example lasting more than one hour
Bluetooth technology for data transmission to the device 5
Electronics for the management of charging via USB of the power supply battery of the same electronics 3
Preferably lithium battery p1 Accelerometer and corresponding electronics for processing the data generated by the accelerometer
Gyroscope and corresponding electronics for processing the data generated by the gyroscope
Time measuring means (clock), preferably included in the microprocessor (not shown) which processes and transmits the data based on the acquired signals

Each measurement session involves a calibration of the insole, which allows to associate a variation of the signal generated by the sensors 2, of the insoles, right and left, to a corresponding variation in pressure.

The device 5 is configured to filter the data relating to the signal generated by each sensor 2 by means of a high pass filter of 0.2 Hz in order to eliminate the contribution due to the weight variation on the foot during the movement (walking) of the operator.

Risk analysis and Assessment Process

The signals from the pressure sensors 2, from the accelerometer 8 and from the gyroscope 9, are acquired and processed by the electronics 3 and are transmitted via a wireless interface to the device 5 or stored on the SD card. The device 5 is configured to analyse the data relating to the signals acquired by the aforementioned sensors, accelerometer and gyroscope and to execute processing algorithms of the same, preferably also taking into account the anthropometric parameters of the operator subjected to the measurements and to generate a customizable report.

The report shows the weight, the number of times, the duration of the handling and the distance travelled with loads.

It is possible to calculate the temporal and cumulative distribution of the movement of a weight greater than one (or more than one) certain threshold, which may vary based on the characteristics of the monitored operator including age, sex, anthropometric data.

In addition to the weight value, the measurement of the accelerometer 8 and of the gyroscope 9 is detected to distinguish the phases of lifting the load only from the phases of handling the load along a path and their duration of the operation.

The report, at the end of each work session, shows:
Number of loads moved
Weight of each load
Duration of the loaded activity
Frequency of repetition of the gesture per unit of time
Distance travelled with load

Method of Implementing the Invention

The device 5 is preferably configured by means of an application running on a smartphone or a personal computer. The application as a user-friendly interface helps the operator to carry out, for each measurement session, the calibration by means of a wizard and the measurements. On the home page, the operator is asked to provide some personal information such as: weight, height, age, sex, information relating to the work being analyzed, indicating for example job, department, work shift, etc., in order to set the threshold values that is the limits of acceptability of the task also in relation to the duration of the same. For example, a weight greater than 23 kg is considered the maximum recommended weight for lifting in ideal conditions, i.e. in an upright position, with the load kept close to the body at 75 cm above the ground, vertical displacement less than 25 cm, optimal grip and low frequency. These conditions are acceptable for 75% of women and over 90% of men (Waters T R, Putz-Anderson V, Garg A, Fine L J (1993). Revised NIOSH equation for the design and evaluation of manual lifting tasks. Ergonomics. 1993 July; 36 (7): 749-76 https://www.cdc.gov/niosh/docs/94-110/default. html). Other references are contained within ISO international standards, for example, ISO 11228-1.

With reference to FIG. 6, the loading of the aforementioned personal information takes place prior to the first block “Static session start”.

Subsequently, wearing the sensors described above, the “calibration” is performed to obtain a correct conversion of weight measurements from electrical signals generated by the sensors. Subsequently, with “Static unloaded measurement”, the weight of the unloaded operator is acquired.

As described above, the signals of the various sensors are averaged and then multiplied by the surface of the insole in which the sensors are inserted to know the actual weight that weighs on each foot of the monitored operator without load. This is equivalent to carrying out a tare measurement.

Subsequently, in “Lifting load”, the weight is monitored with particular reference to any load greater than the aforementioned threshold, preferably 3 kg, by performing the same calculation methods described above, but subtracting the tare calculated in “Static unloaded measurement”.

The measurement of the load is carried out during its lifting. Since the weight is constantly calculated by adding the contribution of both insoles, it is irrelevant that one or both feet are placed on the ground.

When the presence of a load is detected, it is detected in “Load Handling”, through the accelerometer, if the load is maintained in static conditions or if the operator walks.

In this case, various parameters are stored including:
load Lifting/lowering and relative frequency
Load handling, relative duration and distance travelled
Handled weight
Frequency of handling of individual loads in the unit of time and/or in the work session,
Duration of load transportation and distance travelled.

The handled weight can be compared with population-specific reference weight values (sex, age).

It will be possible to identify and calculate:
repetitive movements, for example with a frequency greater than one movement every 5 minutes,
cumulative weight, produced between the handled load and its frequency in the unit of time, in the short (minute) or long (8 hours).

The storage procedure can be interrupted when the load is released to limit the storage of data or it can operate continuously.

Monitoring stops at the end of the “End of session” work session of the monitored operator.

The application allows you to synchronize the wireless interface of the device 5 with the electronics 3 and normalize the response of the sensors 2 with the user's weight.

The raw data files are acquired by the device 5 through the aforementioned wireless interface or through an SD card reader directly connected to the electronics 3.

The data are analysed by device 5 which isolates the activities relevant to the biomechanical risk assessment.

The results of the analysis are shown on the display of device 5 by means of a colour scale that indicates specific threshold levels. These limits are evaluated with respect to the anthropometric characteristics of the operator set at the beginning of the acquisition and measurement session.

The colour scale is selected on the basis of riskiness defined by the reference scientific literature.

The S system can include other wearable sensors or take advantage of information such as GPS location from other devices.

Since the portions of the system directly connected to the body, such as the insoles and the electronics 3, are hermetic and disinfectable, the system can be used by several operators who alternate in work shifts.

The same insoles can be used by users with different foot sizes, as it is known that one insole can be adapted to at least three contiguous foot sizes.

The stability characteristics of the shoe are not altered in any way, therefore, the use of the insoles does not determine the need to certify the footwear in which they are intended to be associated.

The invention can be applied in the field in the workplace without interfering with the work activity, therefore the analysis of the data obtained from the invention allows an assessment of the biomechanical risk related to the handling of loads in the workplace in an objective way.

The invention can be applied in the field in the workplace to workers with different characteristics (age, sex, anthropometric data), therefore the analysis of the data obtained by the invention allows an assessment of the biomechanical risk linked to the handling of loads in the place of specific work for age groups and individual characteristics of the operator, including gender differences.

According to a first operating mode of the system, the acquisitions are stored on the non-volatile memory card SD or similar on board the electronics 3, so that the aforementioned analyzes by the device 5 can be carried out by removing the SD memory card from the device 3 and introducing it into the device 5.

This first operating mode can be useful when it is intended to extend the battery life on board the wearable portion of the system.

According to a second operating mode, the acquisitions are sent, in real time, to the device 5 by means of the aforementioned wireless connection. This mode is more demanding in terms of energy consumption, but allows continuous monitoring of the working conditions of an operator.

According to a further preferred variant of the invention, the data are stored in the non-volatile memory SD, while the communication via wireless connection is used only to signal to the device 5 the exceeding of a predetermined load threshold, for example of 23 kg.

This further operating mode can also be useful when, for example, a plurality of operators is centrally monitored in a monitoring room.

In compliance with the recommendations of ISO international standards (ISO 11228-1. Ergonomics—Manual handling—Part 1: Lifting and carrying), the invention allows to calculate the handled mass and its frequency, the cumulative mass handled daily, the transported mass in relation to the distance travelled and to compare the measured parameters with the recommended reference limits.

Real-Time Identification of Gait Events in Impaired Subjects Using a Single-IMU Foot-Mounted Device—Juan C. Perez-Ibarra, Adriano AG Siqueira, Member, IEEE, and Hermano Igo Krebs, Fellow, IEEE describes a method for calculating a distance travelled on the basis of signals generated by sensors associated with a user's footwear.

Such teachings are implemented here for calculating the distance per stroke by a user while the user carries a load.

In this way it is possible to calculate the work in the unit of time and/or in the work session and the frequency of the transport of loads in the work session. The freight frequency of loads is obtained by comparing the time in which loads are transported over a predetermined time interval, for example equal to one hour and preferably to the entire work session of the operator.

It is worth noting that the calculation of the average value of the loads transported in the work session represents an evaluation parameter other than frequency. In fact, the frequency returns only the number of times a load is lifted over time, without taking into account the weight of the load.

Instead, the evaluation of individual loads as well as their average value returns a much more useful evaluation parameter for assessing the criticality of the work activity.

As described above, according to a preferred variant of the invention, the system S can generate an “alert” by means of the device 5 when the movement of a weight above a certain maximum threshold is detected in real time, which can be set manually or predefined on the basis of the anthropometric characteristics of the operator such as age, sex, weight, height, etc.

The present invention can be advantageously carried out by means of a computer program which comprises coding means for carrying out one or more steps of the method, when this program is executed on a computer. Therefore, it is intended that the scope of protection extends to said computer program and further to computer readable means comprising a recorded message, said computer readable means comprising program coding means for carrying out one or more steps of the method, when said program is run on a computer.

Implementation variants of the described non-limiting example are possible, without however departing from the scope of protection of the present invention, including all the equivalent embodiments, for a person skilled in the art, to the content of the claims.

From the above description, the person skilled in the art is able to realize the object of the invention without the need to introduce further construction details.

1. A system for an assessment of biomechanical risk from handling loads, comprising:
a pair of insoles, each insole comprising a plurality of pressure sensors;
an acquisition electronics for each of the plurality of pressure sensors, wherein the acquisition electronics is arranged to acquire pressure signals generated by the pressure sensors and comprising wireless data interface means arranged to transmit data representative of the generated pressure signals from the pressure sensors;
an analysis and processing device arranged to receive said data and calculate at least one weight value lifted by an operator wearing a pair of shoes in which said pair of insoles is inserted or integrated.

2. The system according to claim 1, wherein said analysis and processing device is further configured to associate a movement duration with a corresponding weight value of a weight transported.

3. The system according to claim 2, further configured to calculate a duration of transportation of all loads of an operator's work session.

4. The system according to claim 3, wherein said operator's work session has a duration of one work shift.

5. The system according to claim 1, wherein said acquisition electronics further comprises an accelerometer and a gyroscopes the data also relates to signals generated by the accelerometer and gyroscope, and the analysis and processing device is arranged to associate the transportation duration of each load and a distance travelled during the transportation itself.

6. The system according to claim 5, wherein the analysis and processing device is further configured to acquire anthropometric information relating to a monitored operator and to calculate a risk index based on the handling of loads carried out during the operator's work session.

7. The system according to claim 6, wherein said acquisition electronics with the relative interface means are included in an impermeable container, and said pressure sensors are impermeable, and is sanitized before use.

8. The system according to claim 1, wherein said acquisition electronics comprises non-volatile storage means for the massive storage of the acquired pressure signals.

9. The system according to claim 1, wherein the acquisition electronics is further configured to autonomously calculate the value of weight of a handled load and transmit data relating to the handling of loads in real time or only when the weight value of a handled load exceeds a predetermined settable threshold.

10. A method for assessing the biomechanical risk by means of the system according to claim 1, comprising:
acquiring a weight value of a load handled by an operator and associating at least a time interval to the weight value,
calculating a handling frequency as load handling time divided by the time of a predetermined time interval and an average value of the weight of the loads handled in the predetermined time interval including the work session in order to report critical conditions in real time.

11. The method according to claim 10, further comprising associating to said weight value of the handled load a corresponding distance travelled during handling and obtaining a work value for each handled load and an overall work in a work session of the operator.

12. The method according to claim 10, further comprising calculating a riskiness index relative to the work session.

13. The system according to claim 5, wherein said analysis and processing device is further configured to associate a movement duration with a corresponding weight value of a weight transported.

14. The system according to claim 13, further configured to calculate a duration of transportation of all loads of an operator's work session.

15. The system according to claim 14, wherein said operator's work session has a duration of one work shift.

16. The system according to claim 8, wherein said analysis and processing device is further configured to associate a movement duration with a corresponding weight value of a weight transported.

17. The system according to claim 16, further configured to calculate a duration of transportation of all loads of an operator's work session.

18. The system according to claim 17, wherein said operator's work session has a duration of one work shift.

19. The system according to claim 8, wherein said acquisition electronics further comprises an accelerometer and a gyroscope, the data also relates to signals generated by the accelerometer and gyroscope, and the analysis and processing device is arranged to associate the transportation duration of each load and a distance travelled during the transportation itself.

20. The system according to claim 19, wherein the analysis and processing device is further configured to acquire anthropometric information relating to a monitored operator and to calculate a risk index based on the handling of loads carried out during the operator's work session.
